(19) United States
(12) Patent Application Publication
(10) Pub. No.: US 2025/0215474 A1
(43) Pub. Date: Jul. 3, 2025

(54) Biosurfactant Producing Microorganisms

(71) Applicant: Dispersa Inc., Laval (CA)

(72) Inventors: Sarah Martinez, Montreal (CA); Nivatha Balendra, Laval (CA); Hela Benaissa, Montreal (CA)

(73) Assignee: Dispersa Inc., Laval, QC (CA)

(21) Appl. No.: 18/852,213

(22) PCT Filed: Mar. 28, 2023
(86) PCT No.: PCT/CA2023/050415

§ 371 Date: Sep. 27, 2024

Related U.S. Application Data
(60) Provisional application No. 63/324,455, filed on Mar. 28, 2022.

(51) Int. Cl. C12P 19/44; C12N 1/16; C12N 1/20; C12P 7/6436; C12R 1/01; C12R 1/645

(52) U.S. Cl. CPC C12P 19/44; C12N 1/165; C12N 1/205; C12P 7/6436; C12R 2001/01; C12R 2001/645

(57) ABSTRACT

There is provided a Burkholderia thailandensis DIS2 bacteria and a Burkholderia thailandensis DIS2.1 bacteria, as well as bacteria having all the identifying characteristics thereof and mutants thereof. There is also provided a Starmerella bombicola DIS4 yeast, and a yeast having all the identifying characteristics thereof and mutants thereof. There is also provided a method for producing rhamnolipids and a method for producing sophorolipids. To produce rhamnolipids, a fermentation is performed with Burkholderia thailandensis, a mutant or variant thereof, with a feedstock comprising a waste carbon source, and fried cooking oil. Similarly, to produce a sophorolipid a fermentation is performed with Starmerella bombicola, a mutant or variant thereof, with a feedstock comprising a waste carbon source, and fried cooking oil.

### TECHNICAL FIELD

This disclosure relates to the field of biosurfactants, more specifically rhamnolipids and sophorolipids, microorganisms producing same, and uses thereof.

### BACKGROUND OF THE ART

Surfactants are surface active agents that can reduce surface tension between two phases, such as liquid-liquid, solid-liquid, and gas-liquid interfaces. These ingredients represent the most versatile category within the chemical industry as they are used in a variety of fields that impact our everyday lives. Surfactants are used in many industries ranging from cleaning products, to cosmetics, agriculture, and more. At industrial scale, surfactants are currently derived from non-sustainable petrochemical processes (i.e. synthetic surfactants) using palm and/or petroleum feedstocks. Synthetic surfactants are divided in two categories: petrochemical surfactants and oleochemical surfactants. Petrochemical surfactants are derived from petroleum feedstock while oleochemical surfactants can be derived from renewable feedstock while still having a portion of palm/petroleum feedstock. Synthetic surfactants pose significant environmental concerns because of their poor biodegradability and notable environmental toxicity. As a consequence, methods of producing biosurfactants, such as rhamnolipids and sophorolipids, are desirable as a sustainable alternative to synthetic surfactants. Biosurfactants can be prepared from palm-based ingredients or can be obtained from microorganisms producing the biosurfactants. Unfortunately, palm tree agriculture is a major cause of deforestation and supports unethical labor standards. Accordingly, it would be more desirable and sustainable to produce biosurfactants from microorganisms.

Additionally, biosurfactants, such as sophorolipids and rhamnolipids, that are currently produced in commercial volumes with microorganisms, are produced using traditional feedstock for fermentation such as lab-grade or food-grade edible feedstocks. It would be much more desirable to produce biosurfactants through non-food grade renewable feedstocks.

Identified for the first time in Pseudomonas aeruginosa cultures, rhamnolipids are among the best-studied biosurfactants. Their excellent surface active properties, low toxicity and high biodegradability make them among the biosurfactants presenting the most commercial potential globally. Due to the pathogenicity of Pseudomonas aeruginosa, non-pathogenic bacteria have been investigated to potentially identify new species for the non-pathogenic production of rhamnolipids. This ultimately has a large impact on the commercialization of such ingredients, as ensuring non-pathogenicity and non-toxicity are key for industries such as consumer products and pharmaceuticals. Burkholderia thailandensis, is one such non-pathogenic bacteria that was identified as a promising candidate for commercial rhamnolipid production. However, current processes employing Burkholderia thailandensis for the production of rhamnolipids have been inefficient, delivering low yields that are commercially nonviable. Apart from rhamnolipids, another biosurfactant that has shown promising commercial potential in recent years are sophorolipids. These biosurfactants are historically produced using yeast species known as Starmerella bombicola. Improvements in the production of biosurfactants, such as rhamnolipids and sophorolipids, particularly in view of industrial scaling and sustainability, are therefore desired.

### SUMMARY

In one aspect there is provided a Burkholderia thailandensis DIS2 bacteria as deposited at IDAC under Accession No. 240222-01, a mutant or a variant thereof. There is also provided a bacteria having all the identifying characteristics of Burkholderia thailandensis DIS2, deposited at IDAC under Accession No. 240222-01, mutants or variants thereof. In a further aspect there is provided a Burkholderia thailandensis DIS2.1 bacteria as deposited at IDAC under Accession No. 070323-01, a mutant or a variant thereof. There is also provided a bacteria having all the identifying characteristics of Burkholderia thailandensis DIS2.1, deposited at IDAC under Accession No. 070323-01, mutants or variants thereof. In still a further aspect, there is provided a Starmerella bombicola DIS4 yeast as deposited at IDAC under Accession No. 210223-01, a mutant or variant thereof. There is also provided a yeast having all the identifying characteristic of Starmerella bombicola DIS4 deposited at IDAC under Accession No. 210223-01, a mutant or variant thereof.

In a further aspect, there is provided a method of producing a rhamnolipid, the method comprising: performing a fermentation with Burkholderia thailandensis, a mutant or variant thereof, wherein the fermentation is performed with a feedstock comprising a waste carbon source, and fried cooking oil. The waste carbon source may be enzymatically treated, physically treated or heat treated or provided untreated. In one embodiment, the waste carbon source is selected from the group consisting of whey, glycerol, maple syrup waste and starch waste. In one embodiment, the fried cooking oil is selected from the group consisting of fried corn oil, fried canola oil, fried peanut oil, fried coconut oil, fried grapeseed oil, fried soybean oil, fried olive oil and fried cottonseed oil. Moreover, ethanol can be further added to the feedstock. The Burkholderia thailandensis can be Burkholderia thailandensis DIS2 or Burkholderia thailandensis DIS2.1. One way of extracting the rhamnolipid is by solvent extraction, such as with ethyl acetate or ethanol as the extraction solvent.

In still a further aspect, there is provided a method of producing a sophorolipid, the method comprising: performing a fermentation with Starmerella bombicola, a mutant or variant thereof, wherein the fermentation is performed with a feedstock comprising a waste carbon source, and fried cooking oil. The waste carbon source may be enzymatically treated, physically treated or heat treated or provided untreated and may be selected from the group consisting of whey, glycerol, maple syrup waste and starch waste. The fried cooking oil can preferably be selected from the group consisting of fried corn oil, fried canola oil, fried peanut oil, fried coconut oil, fried grapeseed oil, fried soybean oil, fried olive oil and fried cottonseed oil. In one embodiment, the Starmerella bombicola is ATCC 22214 or Starmerella bombicola DIS4. One way of extracting the rhamnolipid is by solvent extraction, such as with ethyl acetate or ethanol as the extraction solvent.

Many further features and combinations thereof concerning the present improvements will appear to those skilled in the art following a reading of the instant disclosure.

### DESCRIPTION OF THE DRAWINGS

FIG. 1 is a diagram of an exemplary mutation being introduced in a chromosome of Burkholderia thailandensis.

FIG. 2 is an image of a gel showing the deletion of the scmR gene in Burkholderia thailandensis DIS2.

FIG. 3 is a graph of the optical density measured at 545 nm in function of time for a culture of Burkholderia thailandensis DIS2 in PA14 medium (•), BGR1 medium(▪), and M9 medium (▴).

FIG. 4 is a graph of the optical density measured at 545 nm at the end point of cultures of Burkholderia thailandensis with canola oil, corn oil, ethanol (1.6M or 0.8M), glycerol, sodium gluconate, dextrose, sucrose, mannitol, fructose, maltose, sorbitol, sodium pyruvate, sodium fumarate, sodium acetate, sodium formate, sodium benzoate, sodium octanoate, or sodium succinate.

FIG. 5A is a graph of the optical density measured at 600 nm at the end point of cultures of Burkholderia thailandensis DIS2 with glycerol, sucrose, dextrose, mannitol, sorbitol, corn oil, or ethanol.

FIG. 5B is a graph of the rhamnolipid (RL) production for the cultures of FIG. 5A.

FIG. 6A is a graph of the optical density measured at 600 nm in function of the time for cultures of Burkholderia thailandensis DIS2 at temperatures of 25° C. (•), 30° C. (▪), 32° C. (▴), 35° C. (▾), 37° C. (♦) or 40° C. (∘).

FIG. 6B is a graph of the rhamnolipid (RL) production for the cultures of FIG. 6A.

FIG. 7A is a graph of the rhamnolipid (RL) production for cultures of Burkholderia thailandensis DIS2 with different nitrogen sources: urea, NaNO₃, KNO₃, NH₄NO₃, (NH₄)₂SO₄, or peptone.

FIG. 7B is a graph of the rhamnolipid (RL) production for cultures of Burkholderia thailandensis DIS2 with different phosphate sources: C1 (KH₂PO₄ 0.1M), C2 (KH₂PO₄ 0.05M+K₂HPO₄ 0.05M), C3 (KH₂PO₄ 0.05M+Na₂HPO₄ 0.05M), C4 (K₂HPO₄ 0.1M), C5 (K₂HPO₄ 0.05M+Na₂HPO₄ 0.05M), or C6 (Na₂HPO₄ 0.1M).

FIG. 8 is a graph of the repartition of the factors of the Taguchi analysis performed.

FIG. 9 is a graph of the rhamnolipid (RL) production for each of the sixteen Taguchi factors shown in Table 4.

FIG. 10A is a graph showing the relative dry cell weight (DCW) obtained after a fermentation with nine different oils (one control (oil 1) and eight fried cooking oil (oils 2-9)).

FIG. 10B is a graph showing the relative sophorolipids (SLs) production obtained after a fermentation with nine different oils (one control (oil 1) and eight fried cooking oil (oils 2-9)).

FIG. 10C is a graph showing the relative productivity obtained after a fermentation with nine different oils (one control (oil 1) and eight fried cooking oil (oils 2-9)).

FIG. 11A is a graph showing the dry cell weight (DCW) obtained after a fermentation with glucose, table sugar or waste maple syrup as the carbon source.

FIG. 11B is a graph showing the sophorolipids production obtained after a fermentation with glucose, table sugar or waste maple syrup as the carbon source.

FIG. 11C is a graph showing the productivity of a fermentation with glucose, table sugar or waste maple syrup as the carbon source.

FIG. 12A is a graph showing the dry cell weight after a fermentation with commercial yeast extract (YE1) or waste brewer's yeast extract (YE2 and YE3).

FIG. 12B is a graph showing the sophorolipids production after a fermentation with commercial yeast extract (YE1) or waste brewer's yeast extract (YE2 and YE3).

FIG. 12C is a graph showing the productivity of a fermentation with commercial yeast extract (YE1) or waste brewer's yeast extract (YE2 and YE3).

FIG. 13A shows the biosurfactant profile from a fermentation using pure yeast extract (commercial).

FIG. 13B shows the biosurfactant profile from a fermentation using brewery waste yeast extract.

FIG. 14A is a graph showing the dry cell weight in different fermentations having varying inoculum of yeast.

FIG. 14B is a graph showing the sophorolipids production different fermentations having varying inoculum of yeast.

FIG. 14C is a graph showing the productivity obtained when varying the inoculum of yeast.

### DETAILED DESCRIPTION

There is provided the production of a biosurfactant by a microorganism using agroalimentary waste as feedstock. More specifically, the term “biosurfactant” as used herein refers to surfactant compositions, obtained from microorganisms, comprising or consisting of rhamnolipids or sophorolipids. The microorganisms contemplated by the present disclosure include Burkholderia thailandensis or Starmerella bombicola as well as mutants and variants thereof. In some embodiments, two different mutants of the same strain can be combined. For example, Burkholderia thailandensis DIS2 and Burkholderia thailandensis DIS2.1 (a DIS2 with a ΔscmR mutation) can be combined. In some embodiments, Burkholderia thailandensis E264 (American Type Culture Collection (ATCC) 700388) can be employed for the production of rhamnolipids. The variants include but are not limited to spontaneous mutant strains, mutant strains obtained by ultra-violet or chemical mutagen treatment, cell fusion strains, and genetic recombination strains. In a further example, Starmerella bombicola is employed for the production of sophorolipids. In some embodiments, Starmerella bombicola is Starmerella bombicola DIS4. In further embodiments, Starmerella bombicola is the strain ATCC 22214. The strains contemplated by the present disclosure, particularly DIS2, DIS2.1 and DIS4 are described in further detail herein below.

The present disclosure advantageously utilizes agroalimentary waste as the feedstock for the fermentation performed by the microorganism to produce the biosurfactant. Compared to traditional methods where food-grade edible feedstocks are used, the present method, by replacing the food-grade edible feedstock with agroalimentary waste feedstock, achieves significant cost reductions as well as improved sustainability. The agroalimentary waste of the present disclosure comprises a waste carbon source (treated or untreated) and fried cooking oil. The waste carbon source can be selected from the group consisting of whey, glycerol, maple syrup waste and starch waste. In one example, the waste carbon source can be heat treated. In another example, the waste carbon source can be physically treated such as by grinding. The treatment methods described herein can be combined or performed independently. Whey can be either used “as is” in its waste form as a dairy industry by-product or may be further treated by introducing a lactase enzyme to break down the whey into galactose and glucose. If treated, the whey-derived galactose and glucose can be used in combination or individually for the fermentation. Similarly, starch waste can be provided untreated or can be treated (e.g. enzymatically) to obtain glucose by breaking down the starch polymer. In some embodiments, the maple syrup waste can be provided untreated. In some embodiments, maple syrup waste is defined is the maple syrup discarded during the commercial process as being unfit for consummation (i.e. not edible). This may be due to micro-organism contaminations. In some embodiments, the maple syrup waste is used as obtained without any treatments. Maple syrup waste generally has a high content of sucrose such as from 70 to 90 wt. % or from 75 to 85 wt. %. In some embodiments, the agroalimentary waste used in the present disclosure comprises or consists essentially of whey and fried cooking oil. In some embodiments, the agroalimentary waste used in the present disclosure comprises or consists essentially of maple syrup waste and fried cooking oil. In some embodiments, the agroalimentary waste used in the present disclosure comprises or consists essentially of glycerol and fried cooking oil. Glycerol can be advantageous in the production of sophorolipids.

Biosurfactants such as sophorolipids and rhamnolipids contain fatty acid and sugar parts. A medium comprising both a fatty acid source (e.g. the fried cooking oil) and the waste carbon source was found to advantageously promote the production of biosurfactant. In addition, another advantage is the use of two different sources of waste to improve the environmental benefits of the present process. The fried cooking oil is a frying oil that has been used to fry food and is discarded as waste. The fried cooking oil can be obtained from restaurants, cafeterias, municipalities, waste management, and the like. Examples of fried cooking oil include but are not limited to fried corn oil, fried canola oil, fried peanut oil, fried coconut oil, fried grapeseed oil, fried soybean oil, fried olive oil and fried cottonseed oil. The fried cooking oil can be used in the feedstock as obtained from the waste source, i.e. no pretreatment is necessary. In some embodiments, the fried cooking oil comprises a fatty acid content of from 75 to 85% and a carbon content of 15 to 25%.

A fermentation using a culture of Burkholderia thailandensis mutants or variants thereof, or Starmerella bombicola mutants or variants thereof, is performed to produce the biosurfactant. A pre-culture, if needed, can be performed before the fermentation, on an agar plate and is generally seeded from a stock strain of the microorganism. After 48 hours of incubation on the plate at a suitable temperature (for example around 30° C. for Starmerella bombicola and around 37° C. for Burkholderia thailandensis although 30° C. is also possible for Burkholderia thailandensis), a colony is isolated and replicated for inoculation in a liquid pre-culture in a pre-culture flask. In one example, the pre-culture flask is a 125 mL flask containing 25 mL of agroalimentary feedstock medium. The feedstock can comprise untreated waste carbon source and/or treated waste carbon source, and fried cooking oil. This 48h liquid pre-culture can be used as an inoculum for a larger batch of fermentation.

The fermentation can be performed in a continuous process, a semi-continuous process or in a batch. The medium used for the fermentation can contain from 5 to 20 wt. % of agroalimentary waste (waste carbon source and fried cooking oil). In some embodiments, the medium for the fermentation contains from 7 to 13 v/v %, from 8 to 12 v/v %, from 9 to 11 v/v % or around 10 v/v % of fried cooking oil. In some embodiments, the medium for the fermentation contains 1 to 10 wt. %, 1 to 8 wt. % of waste carbon source (after treatment or before treatment), or 1 to 6 wt. %. In further embodiments, the waste carbon source can be present in a concentration of 20 to 200 g/L, 50 to 150 g/L or about 100 g/L. In some embodiments, the fermentation is performed at a temperature ranging from 25 to 40° C. Although the waste carbon source and fried cooking oil content may be used for a fermentation with Burkholderia thailandensis or Starmerella bombicola, other fermentation parameters such as the temperature, pH, oxygen level, and amounts of salts/nitrogen in the media, will differ since in one case it is a bacterial fermentation and in the other it is a yeast fermentation. In one example, the fermentation is performed as a batch in a 1 L flask for 6 to 8 days at a temperature of 30° C.±5% with Starmerella bombicola or at a temperature of 30° C.±5% with Burkholderia thailandensis and up to 37° C.±5%. The preferred pH for a fermentation with Burkholderia thailandensis is 6±0.3 whereas the preferred pH for a fermentation with Starmerella bombicola is 4±0.5. Other components of the media in the fermentation include a nitrogen source and salts. In some embodiments, the nitrogen source is yeast extract obtained from brewery waste.

The culture media resulting from the fermentation is treated to recover the rhamnolipids or sophorolipids depending on which fermentation was performed (i.e. with Burkholderia thailandensis or Starmerella bombicola). A liquid extraction using an extraction solvent, such as ethyl acetate or preferably ethanol, yields a liquid phase and an organic phase, the organic phase comprising the extraction solvent, and the sophorolipids or rhamnolipids. The liquid extraction can be performed with a ratio of culture media to extraction solvent of 0.8-1.2:1, 0.9-1.1:1, 0.95-1.05:1 or about 1:1 when the extraction solvent is ethyl acetate. The liquid extraction can be performed with a ratio of culture media to extraction solvent of 0.8-1.2:3, 0.9-1.1:3, 0.95-1.05:3, or about 1:3. In some embodiments, the solvent extraction process is repeated until a desirable recovery rate of the rhamnolipids or sophorolipids out of the culture media is achieved. For example, the solvent extraction can be performed one, two, three times or more. The extraction solvent can then be evaporated to obtain the rhamnolipids or sophorolipids in solid form. The rhamnolipids or sophorolipids can then optionally be washed with an organic solvent such as hexane. Hexane can assist in removing fried cooking oil residues. The washing step may be repeated until a desired purity is reached. The product obtained may be re-suspended in a resuspension phase (such as ethyl acetate or ethanol) and then lyophilized and packaged. In some embodiments, the resuspension phase and the extraction solvent have the same composition. The resuspension can be performed in a small amount of resuspension phase relative to the amount of biosurfactant. Other extraction methods are contemplated herein such as by gravimetry or ultrafiltration.

In some embodiments, the Burkholderia thailandensis strain is DIS2 as deposited at the National Microbiology Laboratory, International Depositary Authority of Canada (IDAC), 1015 Arlington Street, Winnipeg, Manitoba, Canada, R3E 3R2, as “Burkholderia thailandensis DIS2”, with Accession Number 240222-01 on Feb. 24, 2022, a mutant or a variant thereof. Burkholderia thailandensis DIS2 was identified by screening a library of thirty strains of Burkholderia thailandensis (also referred to as B. thailandensis) in order to identify a naturally rhamnolipid over-producing strain. The thirty strains were cultivated in tubes containing 5 ml of minimal medium during 7 days, at 30° C. under a 200 rpm agitation. The term “minimal medium” or “minimal media” as used herein is to be understood as is known in the art. In some embodiments, a minimal medium contains the minimal amount of nutrients for a microorganism to grow such as water, a carbon source and inorganic salts. In this case, the minimal medium was a PA14 medium containing (in g/L): 0.9 Na₂HPO₄, 0.7 KH₂PO₄, 2.0 NaNO₃, 0.1 CaCl₂)·2H₂O, 0.4 MgSO₄ 7H₂O, and trace element solution (2 mL/L) (Abdel-Mawgoud et al., 2014). The composition of the trace elements solution was (in g/L): 2.0 FeSO₄·7H₂O, 1.5 MnSO₄·H₂O, and 0.6 (NH₄)₆Mo₇O₂₄·4H₂O. A culture flask was then seeded to confirm the strain selection. At the end of the screening the selected strain was ED852, also named DIS2.

As previously explained, Burkholderia thailandensis produces rhamnolipids. Rhamnolipids are glycolipidic surfactants. Rhamnolipids generally comprise a dimer of 3-hydroxyfatty acids linked through a beta glycosidic bond to a mono- or di-rhamnose moiety, or the rhamnolipids can comprise a single fatty acid chain (instead of the dimer). In some embodiments, rhamnolipids have an alkyl chain of C₈-C₁₂, and may contain one or more unsaturation. Two rhamnolipids exemplary are rhamnosyl-β-hydroxydecanoyl-β-hydroxydecanoate (Rha-C₁₀-C₁₀), a mono-rhamnolipid, and rhamnosyl-rhamnosyl-β-hydroxydecanoyl-B-hydroxydecanoate (Rha-Rha-C₁₀-C₁₀), a di-rhamnolipid.

The yeast Starmerella bombicola was originally discovered in the honey of bumblebees in Canada and in concentrated grape juice in South Africa. Starmerella bombicola was later isolated from the tongue of Bombus pratorum, the early bumblebee, in Spain; and from the body surface of Bombus lapidaries, the red-tailed bumblebee, in Germany. Starmerella bombicola is a yeast sophorolipid producing species. It is a budding yeast with ovoid to ellipsoidal shaped cells. The yeast exhibits diverse colony morphologies that depend on the growth conditions. For example, on malt agar, colonies appear to be small, convex and white. Generally, Starmerella bombicola does not perform any filamentous type of growth. In some embodiments, Starmerella bombicola is the strain provided by the American Type Culture Collection (ATCC): ATCC 22214. ATCC 22214 can be cultured in yeast and mold agar or yeast and mold broth (e.g. ATCC medium 200), yeast extract peptone dextrose (YEPD) (e.g. ATCC medium 1245), and Emmon's modification of Sabouraud's agar (e.g. ATCC medium 28). Starmerella bombicola can grow at temperature ranging from 4° C. to 35° C.

Sophorolipids are surfactants comprising a disaccharide (sophorose) and a w/w-1 hydroxy fatty acid chain. The production quantity and the chemical structure of sophorolipids can depend on the energy source provided to Starmerella bombicola, the pH, the oxygen concentration, as well as other cell culture parameters. Generally, the growth-limiting nutrient of S. bombicola is nitrogen. When nitrogen is depleted, the yeast stops multiplying and enters a ‘stationary phase’ which leads to sophorolipid production. On the other hand, a sufficient amount of phosphorus must be available for the production of sophorolipids. However, nitrogen and phosphorus are not the sole nutrient limitations for the production of sophorolipids. Other nutrient related parameters that can be considered include but are not limited to the C/N or C/P ratio, the absolute amount of glucose or nitrogen in the medium, the type of nitrogen source, the acidity, and the presence of a regulating protein. In preferred embodiments, the Starmerella bombicola strain is Starmerella bombicola DIS4 as deposited at the National Microbiology Laboratory, IDAC, 1015 Arlington Street, Winnipeg, Manitoba, Canada, R3E 3R2, as “Starmerella bombicola DIS4”, with Accession Number 210223-01 on Feb. 21, 2023, a mutant or variant thereof. Details of the identification of Starmerella bombicola DIS4 are provided in the Example section below (see Example 3).

The present disclosure contemplates mutants and variants of Burkholderia thailandensis and Starmerella bombicola. For instance, a mutant of DIS2 with an antibiotic resistance gene was produced, the mutant is referred to herein as Burkholderia thailandensis DIS2.1. Accordingly, in some embodiments, the Burkholderia thailandensis strain is DIS2.1 as deposited at the National Microbiology Laboratory, IDAC, 1015 Arlington Street, Winnipeg, Manitoba, Canada, R3E 3R2, as “Burkholderia thailandensis DIS2.1”, with Accession Number 070323-01 on Mar. 8, 2023, a mutant or a variant thereof.

In view of producing mutants of DIS2, the effect of antibiotics was evaluated on the strain DIS2. Some inhibition tests were performed with trimethoprim, kanamycin and tetracycline. Specifically, a 100 μL of culture of the DIS2 strain was plated in seven different conditions: medium containing 0, 5, 10, 25, 50, 100, or 200 μg/mL of the antibiotics (trimethoprim, kanamycin or tetracycline). The plates were incubated for 48 h at 30° C. After incubation, the growth was evaluated and the minimum inhibitory concentration was determined. DIS2 was able to grow up a concentration of 5 μg/mL of trimethoprim, 200 μg/mL kanamycin and 200 μg/mL tetracycline in the culture.

### Example 1: Burkholderia thailandensis DIS2.1

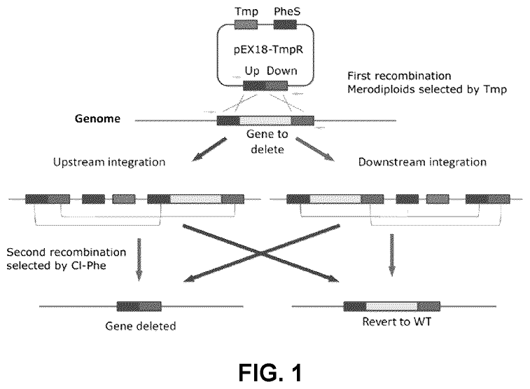

To obtain Burkholderia thailandensis DIS2.1 (“DIS2.1” for short), a gene deletion or mutation was conducted by a two-step allelic exchange. Upstream and downstream of the gene/locus of interest were amplified by polymerase chain reaction (PCR) and inserted into a suicide vector: pEX18 plasmid by Gibson assembly. Then, the constructed vector was transformed into a conjugative E. coli strain: SM10. The mutant allele was then transferred into B. thailandensis (DIS2 strain) by biparental mating between B. thailandensis DIS2 and E. coli SM10. The vector containing the deletion allele was integrated into the B. thailandensis DIS2 chromosome by in vivo homologous recombination and selected against the antibiotic resistance marker (the trimetroprim resistance marker, Tmp was used) encoded within the integrated plasmid backbone. The isolated merodiploid underwent a second homologous recombination, which was counter-selected through the suicide marker PheS (the a-subunit of phenylalanyl tRNA synthase) protein to isolate colonies that have lost the plasmid backbone. In the presence of p-chlorophenylalanine, only strains with disrupted PheS gene are able to grow. As depicted in the FIG. 1, depending on the upstream or downstream integration of the plasmid backbone, the second homologous recombination has in theory a fifty-fifty chance of getting the right confirmation. On the other 50% chances, a wild type revertant is obtained.

For the construction of the suicide pEX18-AscmRup-down-Tmp-PheS vector a PCR was conducted with synthetic oligonucleotides used for cloning which were purchased from Integrated DNA technology. All the PCR reactions were performed with Q5 high-fidelity polymerase (New England Biolabs) in the buffer provided. PCRs of the scmR upstream and downstream regions (900 bp) were realized on the genomic DNA of DIS2 strain using the primer pairs dis63/64 and dis65/66 respectively (Table 1). While the plasmid backbone pET28-Tmp-PheS was amplified in two distinctive sections using the primers dis59/60 and dis61/62 (Table 1) which contain specific overlaps for the insertion of the scmR upstream/downstream sequences into the suicide vector via Gibson assembly.

TABLE 1
Primer sequences with the underlined parts
highlighting the overlap used for the
Gibson assembly.

| | | SEQ |
|---|---|---|
| | | ID |
| Primer | Sequence | NO: |
| Dis59 | TTTCCAACCCGTTGGATCCCCGGGTACCGA | 1 |
| Dis60 | AGTTTTGTGCAATACCAACCGACG | 2 |
| Dis61 | CGTCGGTTGGTATTGCACAAAACT | 3 |
| Dis62 | CGAGGACCATTCCGAAAGCTTGGCACTGGC | 4 |
| | CG | |
| Dis63 | CCAGTGCCAAGCTTTCGGAATGGTCCTCGA | 5 |
| | AGAAGAG | |
| Dis64 | TCTAGAGTCGACCTGCAGGCATGCAAGTAA | 6 |
| | ACTGACGCGCACCTTT | |
| Dis81 | TTTCCAACCCGTTGGATCCCCGGGTACCGA | 7 |
| Dis82 | AGTTTTGTGCAATACCAACCGACG | 8 |

The primers were used at a 0.5 UM concentration each. The PCR program was: 98° C.-2 min, 30 cycles at 98° C., 10 sec; 65° C., 30 sec and 72° C., 30 sec/kb, and finally 72° C.-5 min. All the PCR products were verified on gel agarose and purified using FavorPrep™ GEL/PCR Purification Kit (Favorgen).

For the vector assembly, four different fragments in total (pEX18-1; pEX18-2; ΔscmRup; ΔscmRdown) were combined by Gibson assembly. Isothermal assemblies (Gibson assembly) were performed using homemade mix prepared according to Gibson et al. The resulted assembled vector was purified using FavorPrep™ MicroElute GEL/PCR Purification Kit (Favorgen) and transformed into electrocompetent E. coli DH5a for plasmid maintenance. The plasmid was extracted by FavorPrep™ Plasmid DNA Extraction Mini Kit and confirmed by Sanger sequencing with the M13F and M13R universal primers contained in the pEX18 vector backbone (Centre d′expertise et de services Génome Québec). A final transformation of the confirmed plasmid into electrocompetent E. coli SM10 was done prior to the conjugation with DIS2.

The transformation was performed by incubating two strains together which were spread on Luria-Bertani (LB) plates for an overnight incubation at 37° C. The next day the cells were resuspended in liquid LB medium (at OD_{i-600 nm}=0.05) and incubated at 37° C. under constant agitation until reaching an OD_{600 nm}=0.5-1. The two solutions were then mixed, centrifuged, and resuspended in 50 μL trypticase soy broth (TSB) liquid medium and spread on LB-Agar plates supplemented with trimetroprim antibiotic at 100 μg/mL (for vector pEX18 integration) and gentamycin at 50 μg/mL and polymyxine B at 15 μg/mL (to eliminate E. coli SM10). Only DIS2 strains that have integrated the pEX18 vector into their genome were able to grow. The plates were incubated three days at 37° C. and the colonies were reisolated on fresh LB plates supplemented with the selection marker (trimetroprim at 100 μg/mL).

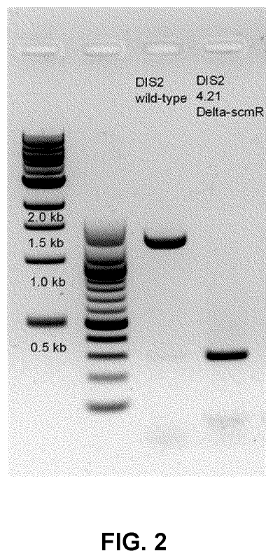

To select the deleted mutants via PheS counter-selection marker, five different colonies were incubated at 37° C. overnight without antibiotic. The next day, different dilutions of the cultures were spread on minimal medium (MM) agar plates supplemented with 10 mM p-chlorophenylalanine for PheS counter-selection. The plates were incubated at 37° C. for five consecutive days. Fifty colonies in total were re-isolated on LB plates with and without trimetroprim to verify the loss of the plasmid backbone (therefore the loss of the antibiotic marker). The sensitive colonies were picked up for PCR colony amplification of the scmR region with the primer pairs dis81/82 (surrounding the scmR gene). Finally, the genomic DNA of the positive strains were extracted and PCRs of the scmR region using the primers 81/82 were conducted and compared to the DIS2 wild-type strain by agarose gel (FIG. 2) and by aligning the sequences. The final selected strain (named DIS2.1) was verified by Sanger sequencing using the dis81 and dis82 primers (Centre d′expertise et de services Génome Québec) (Table 2).

TABLE 2
scmR sequence region of DIS2 and DIS2.1 AscmR.
For DIS2, the scmR gene is highlighted in bold
and underline whereas for DIS2.1, the linker
inserted instead of the scmR gene is in
underlined. The two upstream and downstream
scmR regions are marked with bold font for
both DIS2 and DIS2.1 strains.

| | | |
|---|---|---|
| DIS2 | agatgggaaggctggaaatgcagccggtag | SEQ ID |
| | ccgaacgactggttggcggcatcgaacgaa | No: 9 |
| | tcgtggtcgtactgccggctgctgaaaaga | |
| | atcacgcgcacgcttggcctccggtaaagg | |
| | tgcgcgtcagtttacttcagccgggcgtca | |
| | ctggagcccggcgacgacgtccgggcgctt | |
| | cgccgtgccggcttcgcgcatgaagtagtg | |
| | gccgttcgtctcggtcttctgcccgccgag | |
| | gtggtacagcgcgtgctcgacgaagctgcg | |
| | cgtgcgcgccggcaggaactggcggttcgg | |
| | atagacgatggacaactgcgtgtcgggatc | |
| | gtcgatccggtaatcgccgagaatccgaac | |
| | gagctcgccgctttcgagcgcatcggcgat | |
| | gtagctttccggcagcacggagatgccggt | |
| | gccggcgagcgtcgccgcgcgcaccatcga | |
| | cgcgctgttgaccgtatagaccggctgcag | |
| | cgacacgacgtgcgtgaagtggtccggatc | |
| | gacgaagcgccaggtcgatgcatgctggcc | |
| | ggccggcagcgcgatgcacgcgtggcggat | |
| | gagatcgtcggggcggtgcggcgcgccgtg | |
| | ccgctcgatgtacgagggcgccgcgcagag | |
| | cgcgagcgtgttcggcatcagcggatgacc | |
| | gatgagcgccgggttgccgtcgaggcggcc | |
| | gcccgtgacgaggcccgcgtcgtagccggc | |
| | ttcgatcacgtcgagctgcccctcggtcag | |
| | cgtcaactgcacgcgcaccttcgggtactg | |
| | gcgccggtagctgtcgacgagcggggtgat | |
| | ccggccgggcgacagcagcccggacacggc | |
| | gacgcgcaacgtgccgacgggctcgtgcac | |
| | cgctcgctcgacggacgcctcgagctgatc | |
| | gaattcctcgaggagcgcccggcagccgtc | |
| | gaggtagcggatgcccgcttccgtcagcga | |
| | cagattgcgtgtcgtgcgatggatgagacg | |
| | cgtgttcagatgtgtttcgagcatcgcgat | |
| | cgaacgcgtgacgagcgcattcgatacgcc | |
| | gagtttccgcgccgcccggcggaagctctg | |
| | cagttcggcaacacatacgaagacacgcat | |
| | ggtctggatttggttcatagctttcgttgg | |
| | cccattaaatgattatgttgtgtttttcgc | |
| | cttcgatcatggcgagccgcggctgtcgga | |
| | tcagcacagggcttgtcttaaataacgaat | |
| | catcgaaggctgggcaaattgttcaataag | |
| | ctgacagcatcagtca | |
| DIS2.1 | nnnggggcgcagaggttggggctcgacgat | SEQ ID |
| | cgtggtcgtactgccggctgctgaaagaat | No: 10 |
| | cacgcgcacgcttggcctccggtaaaggtg | |
| | cgcgtcagtttacttgcatgcctgcaggtc | |
| | gactctagaagctttcgttggcccattaaa | |
| | tgattatgttgtgtttttcgccttcgatca | |
| | tggcgagccgcggctgtcggatcagcacag | |
| | ggcttgtcttaaataacgaatcatcgaagg | |
| | ctgggcaaattgttcaataagctgacagca | |
| | tcagtcaaa | |

### Example 2: Optimization of Burkholderia thailandensis Growth Conditions

Three steps are necessary for the implementation of Taguchi methods. Designing the experiment represents the first step, then the experiments are performed and analyzed, and finally a validation assay is accomplished to confirm the predictive model (Morsi et al., 2004). Taguchi methods are based on the use of orthogonal arrays, which allows the distribution of the variables, also named factors, in a balanced manner.

In the present example, a first step of optimization, aiming at the identification of a minimal medium which allows the production of rhamnolipids was performed. Different carbon, nitrogen and phosphorous sources were screened in minimal medium composition in order to select the best candidates for the optimized production medium. Subsequently, the optimized temperature for rhamnolipid production was determined. Finally, a Taguchi method was implemented to identify the best conditions for the optimization of rhamnolipid biosynthesis in B. thailandensis DIS2.

Three minimal salt media were tested based on mineral media described for rhamnolipid biosynthesis by other bacteria, such as Pseudomonas aeruginosa or Burkholderia glumae. The first media tested was M9 medium which contained (in g/L): 15 KH₂PO₄, 64 Na₂HPO₄, 0.1 CaCl₂), 2.0 MgSO₄·7H₂O, 2.5 NaCl and 13.78 NaNO₃ (Smith et al., 2016). The second media tested was BGR1 medium which contained (in g/L): 3.67 KH₂PO₄, 2.643 K₂HPO₄, 0.13 CaCl₂), 0.1 MgSO₄ 7H₂O and 9 urea (Nickzad et al., 2018). Finally, the third media tested was PA14 medium containing (g/L): 0.9 Na₂HPO₄, 0.7 KH₂PO₄, 2.0 NaNO₃, 0.1 CaCl₂)·2H₂O, 0.4 MgSO₄ 7H₂O, and trace element solution (2 mL/L) (Abdel-Mawgoud et al., 2014). The composition of the trace elements solution was (g/L): 2.0 FeSO₄·7H₂O, 1.5 MnSO₄·H₂O, and 0.6 (NH₄)₆Mo₇O₂₄·4H₂O. The experiments were realized in micro-plates: 200 μL of medium were inoculated with a starting OD₆₀₀ of 0.2. The plates were incubated in a Bioscreen™ (Growth Curves, USA) for 72 h at 37° C.

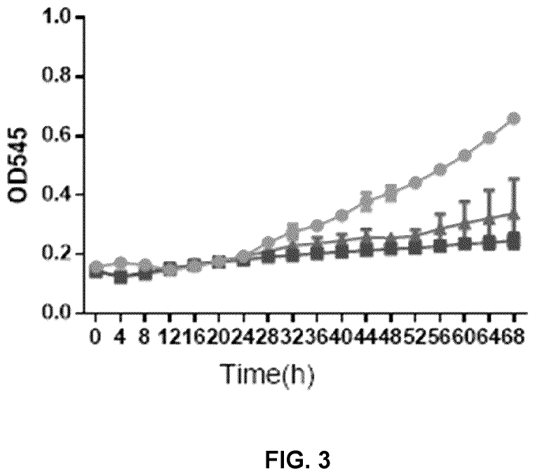

In order to define a minimal medium allowing the growth of B. thailandensis, cultures were performed in five replicates with the three different minimal medias complemented with 4% glycerol. Growth curves obtained from the Bioscreen™ revealed that the PA14 medium was the best candidate for B. thailandensis cultivation out of the three tested medium (FIG. 3).

Several carbon sources were pre-selected for testing: glycerol, sucrose, dextrose, mannitol, sorbitol, ethanol, canola oil, fructose, corn oil, sodium gluconate, sodium pyruvate, sodium succinate, sodium octanoate, sodium fumarate, sodium benzoate, sodium acetate and sodium formate. The carbon sources were added in the PA14 medium at a concentration fixed at 1.6M, except for the corn oil. Medium was supplemented with 4% (w/v) of corn oil. A lower concentration of ethanol (0.8M) was also tested, due to the toxicity of this compound for B. thailandensis.

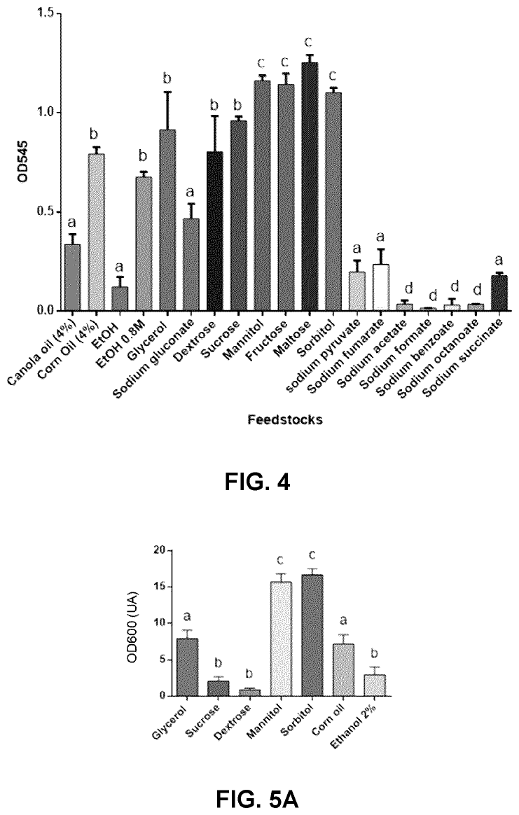

Cultures were performed in the PA14 medium with different carbon sources at 37° C. during four days in Bioscreen™. Agitation was performed for 20 seconds before measuring the optical density at 545 nm (OD₅₄₅) values (FIG. 4). The statistical difference between the data points was determined using a one-way analysis of variance (ANOVA) followed by Duncan's multiple range test. Different lower-case letters indicate statistically significant differences (p<0.05). Results identified with the same letter were not found to be statistically different. Results with different letters were found to be significantly different. The error bars indicate the standard error of the mean (n=3).

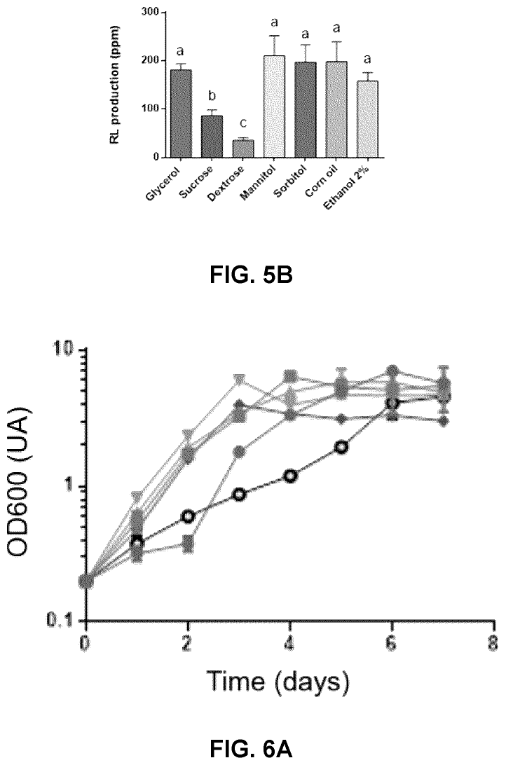

From FIG. 4, the seven of the best performing carbon sources were selected: glycerol, sucrose, dextrose, mannitol, sorbitol, corn oil, and ethanol 0.8 M (or 2 wt. %) for further testing. New cultures were performed in tubes containing 3 mL of minimal medium PA14 during 5 days. The cultures of B. thailandensis were again performed with the same concentration of carbon sources (i.e. 1.6 M except for ethanol (0.8M) and corn oil (4% v/v)). At the end of the cultivation period, the OD₆₀₀ and rhamnolipid production were measured (FIGS. 5A and 5B respectively). The statistical difference was determined using an ANOVA followed by Duncan's multiple range test. Different lower-case letters indicate statistically significant differences (p<0.05). Results identified with the same letter were not found to be statistically different. Results with different letters were found to be significantly different. The error bars indicate the standard error of the mean (n=3).

The OD₆₀₀ values were better when B. thailandensis grew on mannitol or sorbitol (FIG. 5A), however the OD₆₀₀ can be biased by the polyhydroxyalkanoate (PHA) content in cells (Martinez 2020). Therefore, a direct conclusion could not be drawn at this point if the differences seen depend solely on the growth of bacteria or if the PHA production has an impact on the OD₆₀₀ values. Moreover, the rhamnolipid production was similar for cultures performed in glycerol, mannitol, sorbitol, corn oil or ethanol (FIG. 5B). Based on economic considerations, assuming equal or similar rhamnolipid production levels, the two optimal carbon sources are glycerol and ethanol. Ethanol is affordable and could represent an asset for fermentations because of its anti-foam property which is advantageous for the mixing in fermentations. For these reasons, ethanol was selected for the subsequent experiments.

A range of temperatures for the culture media was tested: 25, 30, 32, 34, 37 and 40° C. The two boundaries represent typical minimal and maximal temperatures used in the industry. Experiments were performed in 4 replicates in 125 mL-baffled flasks containing 25 mL of minimal medium (PA14) with 2% ethanol during 6 days under a 200 rpm rotary agitation (Infors™). Rhamnolipids were quantified in one final point. The experiment was repeated two times, and one-way analysis of variance (ANOVA) was performed to evaluate the significance of each carbon or nitrogen source on the rhamnolipid production for the statistical confidence of 95%.

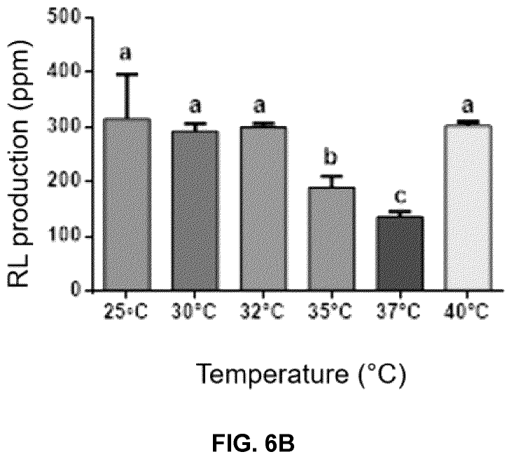

The results of the preliminary screening for various culture temperatures are shown in FIGS. 6A and 6B. OD₆₀₀ values are presented in FIG. 6A. In all the tested growth temperatures the OD₆₀₀ reached the same value, around 6 UA. However, the growth rate is different depending on the temperature. Indeed, B. thailandensis grew more slowly at 25° C. and 40° C., with a stationary phase reached in 6 days compared to the others conditions for which the length of the logarithmic phase was 3 or 4 days. Among the six temperatures tested, rhamnolipid production was highest when B. thailandensis was cultured at 25° C., 30° C., 32° C. or 40° C. (FIG. 6B). Cultures were performed in PA14 medium containing EtOH as carbon source, during seven days, with an agitation at 200 rpm. Statistical difference was determined using an ANOVA followed by Duncan's multiple range test. Different lower-case letters indicate statistically significant differences (p<0.05). The error bars indicate the standard error of the mean (n=4). The values are means±standard deviations (error bars) for four replicates.

A previous study carried out on B. thailandensis demonstrated that the rhamnolipid production is prevented by higher temperatures, which favor PHA production. Indeed, rhamnolipid production reached 2.79 g/L and 1.99 g/L at 25° C. and 30° C. respectively, when B. thailandensis is cultured in shake flasks experiments during 264h in NB medium with 4% glycerol (Funston et al., 2016). In the present tested conditions, a decrease of the rhamnolipid production was observed when the temperature became higher (35° C. and 37° C.), with 189 ppm and 136 ppm. The composition of the medium was minimal in the present example versus rich for Funston et al.'s study (Funston et al., 2016), which could explain the lag observed for the range of temperature decreasing the rhamnolipid production. Surprisingly, the rhamnolipid production increased when the cultures were performed at 40° C.

Based on the OD₆₀₀ and rhamnolipid values obtained, the temperature of 30° C. was selected as the optimal temperature for several reasons. Rhamnolipid production was the best for cultures performed at 25° C., 30° C., 32° C. or 40° C., but growth was slower for the extreme temperatures. So in order to develop the shortest and cheapest process, a temperature of 30° C. was selected for the subsequent experiments.

The nitrogen and phosphate sources were then studied. Specifically, the following six nitrogen sources were studied: urea, NaNO₃, KNO₃, NH₄NO₃, (NH₄)₂SO₄ and peptone. They were added to a minimal medium PA14 based on a fixed molarity (calculated from the molarity of NaNO₃ in the minimal medium first tested): 20 mM. Six combinations of three phosphates (KH₂PO₄, K₂HPO₄, Na₂HPO₄) sources were studied here (shown in Table 2). They were added to a minimal medium PA14 based on a fixed molarity (calculated from the molarity of the combination of KH₂PO₄ and Na₂HPO₄ in the minimal medium first tested): 0.1 M. The effects of various nitrogen and phosphorus sources were tested by using ethanol as the carbon source. Experiments were performed in 125 mL-baffled flasks containing 25 mL of MSM medium with 2% glycerol during 6 days under a 200 rpm rotary agitation (Infors™). N-2-hydroxyethylpiperazine-N-2-ethane sulfonic acid (HEPES) at pH=7 was added (200 mM) in the medium before inoculation at OD₆₀₀=0.05. Rhamnolipids were quantified in one final point. The experiment was repeated two times, and one-way analysis of variance (ANOVA) was performed to evaluate the significance of each nitrogen or phosphorus source on the rhamnolipid production for the statistical confidence of 95%.

TABLE 2
Phosphates concentration used for the different tested combinations

| | Combi- | Combi- | Combi- | Combi- | Combi- | Combi- |
|---|---|---|---|---|---|---|
| | nation 1 | nation 2 | nation 3 | nation 4 | nation 5 | nation 6 |
| | (C1) | (C2) | (C3) | (C4) | (C5) | (C6) |
| KH₂PO₄ | 0.1M | 0.05M | 0.05M | / | / | / |
| K₂HPO₄ | / | 0.05M | / | 0.1M | 0.05M | / |
| Na₂HPO₄ | / | / | 0.05M | | 0.05M | 0.1M |

Cultures were performed in PA14 medium with fixed concentration of EtOH (0.8M), at 30° C. during five days, with an agitation at 200 rpm. Nitrogen sources were added at 20 mM except for the peptone, which was added at a standard concentration used in industry (5 g/L). Phosphorus sources were added as represented in table 2. The statistical difference was determined using an ANOVA followed by Duncan's multiple range test. Different lower-case letters indicate statistically significant differences (p<0.05). Results identified with the same letter were not found to be statistically different. Results with different letters were found to be significantly different. The error bars indicate the standard error of the mean (n=4).

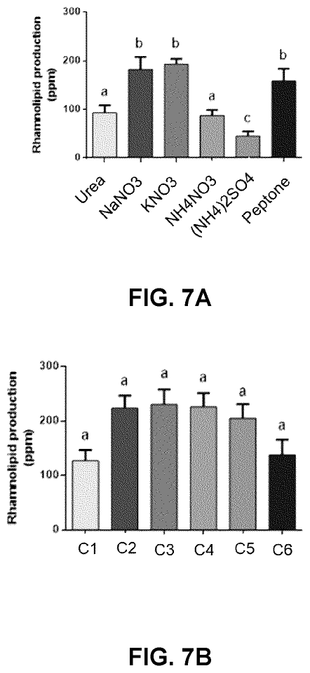

The results of the preliminary screening for various nitrogen and phosphorus sources are shown in FIGS. 7A and 7B. Among the six nitrogen sources tested, a significant difference was observed for the rhamnolipid production when the cultures were performed with NaNO₃ (FIG. 7A). However, surprisingly the cultures tested with urea underperformed considering urea is known as the optimal nitrogen source for the rhamnolipid production in B. glumae. Regarding the phosphate buffer composition, none of the six combinations tested had a significant effect on the rhamnolipid production (FIG. 7B).

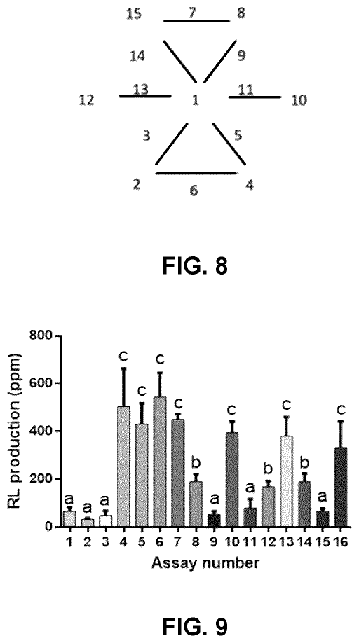

Based on these observation NaNO₃ and the combination 3 of phosphorous salts were chosen for the next experiments. The value of rhamnolipid production was 230 ppm under non optimized conditions. A set of fractional factorial designs was used for the Taguchi experimental plans, consisting of an orthogonal array, which concentrate on main effect estimation. The design of experiments using the orthogonal array drastically reduces the number of experiments to be performed. In the present study, Taguchi tables were used and more specifically a L16 design, which allowed to study up to fifteen parameters with 16 runs, including some interactions studies (FIG. 8). For each factor, two values can be evaluated. Several configurations are available for L16 Taguchi designs. In the present model, each number corresponds to an effect. More precisely, a number above or below a line actually represents an interaction between the two factors corresponding to the numbers on both sides of the line. For instance, the factor number 3 gives the response of the interaction between the factors 1 and 2. The factors were distribute the to minimize the probability of interactions between the factors. Based on this strategy the factors are summarized in Table 3 below.

TABLE 3
Taguchi parameters

| Factor | Distribution number |
|---|---|
| HEPES | 1 |
| Carbon | 2 |
| HEPES/Carbon | 3 |
| NaNO₃ | 4 |
| HEPES/NaNO₃ | 5 |
| Carbon/NaNO₃ | 6 |
| MgSO₄/phosphate | 7 |
| MgSO₄ | 8 |
| HEPES/MgSO₄ | 9 |
| TES | 10 |
| HEPES/TES | 11 |
| CaCl₂ | 12 |
| Yeast extract (YE) | 13 |
| HEPES/phosphate | 14 |
| Phosphate | 15 |

Sixteen runs were performed as described below, for B. thailandensis E264 strain (Table 4).

TABLE 4
Experimental design of the Taguchi table (“−” = absence; “+” = presence)

| | HEPES | EtOH | NaNO₃ | MgSO₄ | TES | CaCl₂ | YE | Phosphates |
|---|---|---|---|---|---|---|---|---|
| N assay | (200 mM) | (M) | (mM) | (mM) | (2 ml/L) | (mM) | (5 g/L) | (mM) |
| 1 | − | 0.4 | 20 | 1.6 | − | 0.9 | − | 25 |
| 2 | − | 0.4 | 20 | 3.2 | + | 1.8 | + | 50 |
| 3 | − | 0.4 | 100 | 1.6 | − | 1.8 | + | 50 |
| 4 | − | 0.4 | 100 | 3.2 | + | 0.9 | − | 25 |
| 5 | − | 0.8 | 20 | 1.6 | + | 0.9 | − | 50 |
| 6 | − | 0.8 | 20 | 3.2 | − | 1.8 | + | 25 |
| 7 | − | 0.8 | 100 | 1.6 | + | 1.8 | + | 25 |
| 8 | − | 0.8 | 100 | 3.2 | − | 0.9 | − | 50 |
| 9 | + | 0.4 | 20 | 1.6 | − | 0.9 | + | 50 |
| 10 | + | 0.4 | 20 | 3.2 | + | 1.8 | − | 25 |
| 11 | + | 0.4 | 100 | 1.6 | − | 1.8 | − | 25 |
| 12 | + | 0.4 | 100 | 3.2 | + | 0.9 | + | 50 |
| 13 | + | 0.8 | 20 | 1.6 | + | 0.9 | + | 25 |
| 14 | + | 0.8 | 20 | 3.2 | − | 1.8 | − | 50 |
| 15 | + | 0.8 | 100 | 1.6 | + | 1.8 | − | 50 |
| 16 | + | 0.8 | 100 | 3.2 | − | 0.9 | + | 25 |

Level average analysis, is one of the techniques used to explore the results of the Taguchi methods: the average effect of each factor is determined on the outcome of the experiment. Hence the factors that have the strongest effects are identified.

The equation below shows the method of calculating the average effect of the experiment:

$$T = ( {Y}_{1} + {Y}_{2} + … + {Y}_{n} ) {/}_{n}$$

with Y₁=result of the first experiment, Y₂=result of the second experiment, T represents the overall average of the experiment, and n is the number of the experimental runs.

For example, for the calculation of the effect of the two values of factor A, A1 and A2 represent the average effect of factor A for levels 1 and 2, respectively. The equations below allow the evaluation of the effect for each level.

$$\begin{matrix}A 1 = ( {Y}_{1} + {Y}_{2} + … + {Y}_{8} ) / 8 \\ A 2 = ( {Y}_{9} + {Y}_{10} + … + {Y}_{16} ) / 8\end{matrix}$$

For each factor, the relative impact (ΔX) is calculated as the difference between the highest and the lowest average response of each level. By this way the effects of all factors, allowing the identification of the factors with the strongest effect. The experimental design for 16 runs at two levels for each factor along with mean values of responses for three replicates (±standard deviation) is presented in FIG. 9. The sixteen runs were performed at 30° C. during 5 days with an agitation at 200 rpm. The statistical difference was determined using an ANOVA followed by Duncan's multiple range test. Different lower-case letters indicate statistically significant differences (p<0.05). The error bars indicate the standard error of the mean (n=3).

Data sets of responses for rhamnolipid production were explored to identify optimum operating conditions. Based on the obtained responses for each run, the effect of each factor was calculated following the level average analysis. The mean value of all runs is 245 ppm. Detailed results describing the effect of each factor are presented in Table 5.

The first observation that can be made here is that the pH is important for rhamnolipids production if B. thailandensis E264. Indeed, when 200 mM HEPES buffer was added in the culture medium, causing a stabilization of the pH during the culture at a value of 7.00, rhamnolipid production was increased.

The concentration of the carbon source had an impact too, since a better production of rhamnolipid was obtained when B. thailandensis was cultured with 0.8M ethanol instead of 0.4M. Ethanol was already used as feedstock for rhamnolipid production in P. aeruginosa, allowing a better rhamnolipid production compared to other substrates such as glycerol, rape seed oil or glucose. It was desired to evaluate if ethanol could be a better substrate for increasing the rhamnolipid production because it could be used as an antifoam and therefore represents an advantage for the implementation of an industrial scale process of biosurfactant production.

The addition of yeast extract in the medium does not play an important role for the rhamnolipid biosynthesis. The use of yeast extract as nitrogen source was already studied in P. aeruginosa, resulting in a reduction of the rhamnolipid production. It was desired to test the addition of yeast extract in order to reduce the lag phase in the cultures performed in minimal medium. Since that seems to not affect the rhamnolipid production, the use of yeast extract in a small concentration was considered.

On the other hand, the addition of trace element solution promoted the production. Interestingly, the opposite effect has been observed in B. glumae. Indeed a recent study showed that the trace elements (metallic ions) positively affected the bacterial growth but prevent the rhamnolipid biosynthesis (Nickzad et al., 2018).

Then the limitation in NaNO₃, CaCl₂) and phosphate promoted the rhamnolipid pathway since better results at the low level corresponding to these factors were obtained. Surprisingly, the present results revealed that the higher concentration of MgSO₄ allowed a better production of rhamnolipid in B. thailandensis. In contrast with that, the optimization work for rhamnolipid production in B. glumae demonstrated that MgSO₄ did not affect the biosurfactant synthesis (Nickzad et al., 2018).

TABLE 5
Effect of each factor in the L16 design on the rhamnolipid production
by B. thailandensis E264

| | HEPES | Carbon | NaNO3 | MgSO4 | TES | CaCl2 | YE | Phosphorus |
|---|---|---|---|---|---|---|---|---|
| Level | −33.73 | −77.25 | 15.92 | −48.94 | −78.58 | 20.07 | −5.61 | 98.3 |
| 1 | | | | | | | | |
| Level | 33.73 | 77.25 | −15.92 | 48.94 | 57.75 | −20.07 | 5.61 | 12.59 |
| 2 | | | | | | | | |

Unfortunately, it seems that some significant interactions between factors are present in the present model, judging by the effect that can be attributed to the other columns of the matrix (Table 6). Indeed, if no interaction was detected, the values associated for each column would be close to zero. Instead, a major interaction for the columns 3 (HEPES/Carbon), 5 (HEPES/NaNO₃), 6 (Carbon/NaNO₃) and 7 (MgSO₄/phosphate) was observed.

TABLE 6
Effect associated to the factor corresponding to interactions
in the chosen Taguchi's design.

| Column | 3 | 5 | 6 | 7 | 9 | 11 | 14 |
|---|---|---|---|---|---|---|---|
| Level 1 | −43.29 | −30.44 | −69.73 | 51.18 | 14.35 | −13.45 | 9.58 |
| Level 2 | 43.29 | 30.44 | 47.79 | −51.18 | −14.35 | 13.45 | −9.58 |

After the impact of each factor on the rhamnolipid production was determined, their combined effect was calculated since the effects of each factor is cumulative in the Taguchi's methods. By this way a new experiment is designed depending on the results generated by the L16 table. This experiment was carried out in order to validate the model based on the assumptions.

The best level for each factor was selected, giving a theoretical value based on the fact that the individual effects are cumulative in this kind of experiments. The values in bold in Table 7 represent the level of each factor leading to an enhancement of the rhamnolipid production.

TABLE 7
Combinations for the optimization of rhamnolipid production

| | HEPES | EtOH | NaNO₃ | MgSO₄ | TES | CaCl₂ | YE | Phosphorus |
|---|---|---|---|---|---|---|---|---|
| Level | −33.73 | −77.25 | 15.92 | −48.94 | −78.58 | 20.07 | −5.61 | 98.3 |
| 1 | | | | | | | | |
| Level | 33.73 | 77.25 | −15.92 | 48.94 | 57.75 | −20.07 | 5.61 | 12.59 |
| 2 | | | | | | | | |

To validate the optimization results determined by the experiment, a shake flask experiment was carried out under the optimum culture conditions (Table 7) and the production of rhamnolipids was determined. The observed experimental values (mean of triplicates) and values predicted by the equations of the models are presented in Table 8. The confirmation run did not allow to reach the predicted value of rhamnolipid production, suggesting that some interactions take place between the factors. However, the implementation of the Taguchi's table and more precisely the L16 table demonstrated that this method is effective because a higher value for rhamnolipid production was obtained compared to the initial conditions studied.

TABLE 8
Experimental values of rhamnolipid production

| | Value before | | |
|---|---|---|---|
| Response | optimization | Predicted value | Mean experimental value |
| RL (ppm) | 230 | 817.15 | 557.52 |

### Example 3: Optimization of the Sophorolipid Production

Sophorolipid production depends on the presence of sugars and fatty acids as feedstocks for the yeast. The culture medium used contained 100 g/L glucose, 10% (v/v) oil, 5 g/L yeast extract, 0.7 g/L pepton, 0.7 g/L salts. Several feedstocks were tested and the sophorolipid production was confirmed when S. bombicola ATCC 22214 was cultured using whey, glycerol, and used cooking oil. The whey is a milk industry derived waste, containing a large amount of lactose. Structurally speaking, the lactose is a sugar composed by one glucose unit and one galactose unit. A pre-treatment of the lactose using a specific enzyme named lactase allowed the liberation of the glucose unit. Subsequently the yeast Starmerella bombicola can use the glucose and/or the galactose as a feedstock. Glycerol is a by-product from bioethanol production by fermentation and can also be considered as a potential feedstock for the sophorolipid production. Glycerol improves the acidic form of sophorolipids.

Flask assays revealed the potential of 3 feedstocks for the sophorolipid (SPL) biosynthesis as described in the table below. SPL production was determined gravimetrically.

TABLE 9
SPL production results:

| Feedstock | Production of SPL in flask (g/L) |
|---|---|
| Pre-treated whey + Waste cooking oil | 5.97 |
| Pure Glycerol + Waste cooking oil | 35.4 |
| Pure Glucose + Waste cooking oil | 33.9 |

The impact of the initial pH was estimated by comparing cultures performed with a starting pH fixed a 4 or at 6.5. The results indicated that an adjusted pH at 4 at the beginning of the cultivation led to a better production of sophorolipid. This result was obtained in flask first and then confirmed in a bioreactor. These tests were realized using glucose (100 g/L) and waste cooking oil (10%) as feedstocks are summarized in Table 10.

TABLE 10
Results for the pH optimization

| | |
|---|---|
| pH adjusted at 4.5 | Unadjusted pH |

| | | | | |
|---|---|---|---|---|
| Cultures in flasks | 18.9 | g/L | 14.2 | g/L |
| Cultures in bioreactor | 162 | g/L | 116 | g/L |

### Example 4: Starmerella bombicola DIS4

A collection of approximately 50 bumblebees species (Bombus impatiens) were collected during May/June 2022 (from Ontario and Quebec, Canada). The bumblebees were then frozen, thawed and crushed into a phosphate saline (PBS) solution buffer. The different solution stocks were stored at −80° C. in 30% glycerol (v/v). Different volumes of the extracted solutions were plated on 239×239 cm² petri dishes containing all the water-soluble nutrients for sophorolipids production and supplemented with a chloramphenicol antibiotic (33 μg/mL final concentration) to inhibit bacterial growth. After three days of growth, a vaporization test was performed using atomized mineral oil assay as described in Burch et al., 2010, with a few modifications (addition of Sudan Red). Sudan red was added to mineral oil to provide a better contrast (500 mg/100 mL). The presence of a halo surrounding colonies indicated the production of sophorolipids caused by the amphiphilic properties of surfactants; the diameter of the halo was measured and compared to the Starmerella bombicola 22214 ATCC control strain. All the halo-producing colonies were isolated, cultured and plated back for a second oil vaporization confirmation test.

Genomic DNA of the strains were also extracted to identify the nature of the strain species. The D1/D2 domain region of the large subunit of rDNA (16S) were amplified by Polymerase Chain Reaction using the standard Taq DNA Polymerase (New Englands Biolabs) using the NL (SEQ ID No. 11: GCATATCAATAAGCGGAGGAAAAG)/NL4 (SEQ ID No. 12: GGTCCGTGTTTCAAGACGG) primers. The PCR program was: 95° C. for 30 sec, 30 cycles at 95° C. for 30 sec; 50° C. for 1 min then 68° C. for 1 min, and finally 68° C. for 5 min. Following the PCR reaction, the products were verified on agarose gel and purified with a MicroElute™ PCR cleanup kit (Favorgen™). The D1/D2 PCR products were sequenced by Sanger (Genome Quebec). All the obtained sequences were identical to the D1/D2 subunit of the S. bombicola 22214 ATCC strain, thus belonging to the Starmerella clade. The strain showing the highest sophorolipids production (with growth rate comparable to the ATCC strain), was picked up for full genomic next-generation sequencing by Illumina sequencing technology (Seqcenter). Analysis of the sequences were performed on Geneious™ Prime software and mapped to the sixteen contiguous sequences of the ATCC 22214 strain as a reference, giving a % pairwise identity ranging from 98.6 to 91.2%. The strain selected was named DIS4.

### Example 5: Use of Waste Fry Cooking Oil in the Production of Sphorolipids

Flask assays were conducted in 125 mL baffled flasks (25 mL of working volume) in triplicate to test pure canola oil (oil 1=pure canola oil as control) compared to eight different waste cooking oils, oils 2 to 9 which are a mixture of different waste frying oils containing mainly canola oil obtained from local restaurants (Quebec, Canada). For each condition, the tests in flasks were realized using pure glucose (100 g/L), commercial yeast extract (5 g/L) and a mix of salts containing KH₂PO₄ (1 g/L), MgSO₄, 7H₂O (0.5 g/L), CaCl₂), 2H₂O (0.1 g/L) and NaCl (0.1 g/L). The pH was also adjusted to 4 (with the addition of HCl solution at 2M) prior to the addition of the different oils. For each assay, the different sources of oil were added at a final 10% (v/v) concentration at the beginning of the cultivation. All the flasks were inoculated at OD_{i,600 nm}=0.1 starting from an overnight pre-culture of S. bombicola and incubated at 30° C. for seven constitutive days under constant shaking (200 RPM).

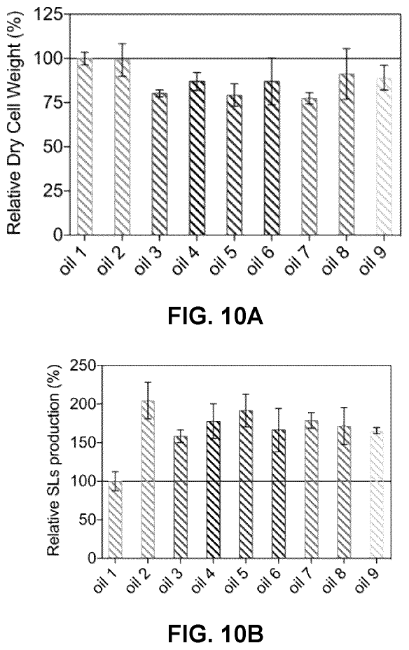

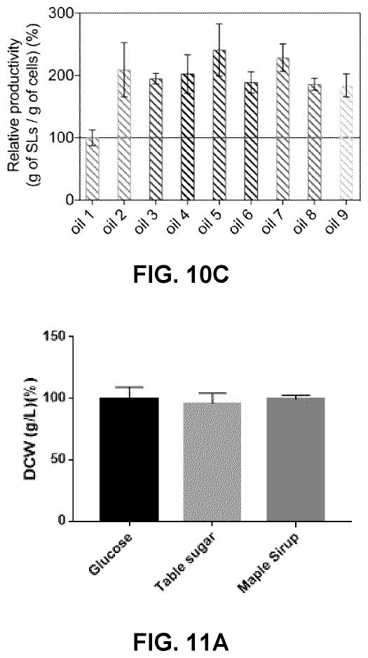

As shown in FIGS. 10A-10C, waste frying oil was surprisingly found to outperform pure canola oil. With a similar dry cell weight obtained (FIG. 10A), the sophorolipids production and relative productivity (FIGS. 10B-10C) were higher. Therefore, in addition to being an attractive economic and environmental choice, fried cooking oil was shown to improve the productivity of biosurfactant.

### Example 6: Use of Maple Syrup Waste as a Carbon Source in the Fermentation

Waste maple syrup was collected from the “Association des Producteurs et Productrices des acericoles du Quebec” as a declassified maple syrup (inconsistent for the alimentation). Flask assays were conducted in 125 mL baffled flasks (25 mL of working volume) in triplicate to test waste maple sirup compared to table sugar (sucrose) since maple syrup presents a high content of sucrose (around 80% of sucrose). As a control, table sugar (food grade) was used as a sugar source. For each condition, the tests in flasks were realized using waste fried cooking oil at 10% v/v, commercial yeast extract (5 g/L) and a mix of salts containing KH₂PO₄ (1 g/L), MgSO₄, 7H₂O (0.5 g/L), CaCl₂), 2H₂O (0.1 g/L) and NaCl (0.1 g/L). The pH was also adjusted to 4 (with the addition of HCl solution at 2M) prior to the addition of the oil. All the flasks were inoculated at OD_{i, 600 nm}=0.1 starting from an overnight pre-culture of S. bombicola DIS4 and incubated at 30° C. for seven constitutive days under constant shaking (200 RPM).

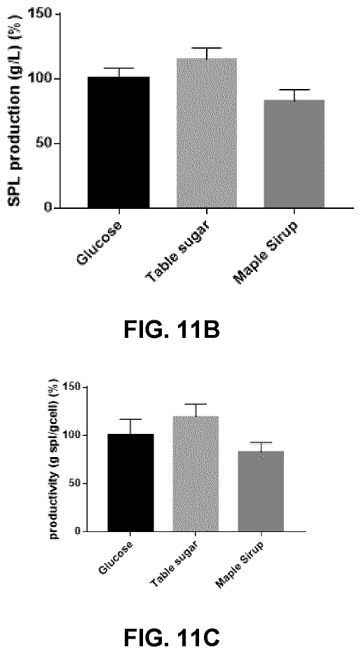

As shown in FIGS. 11A-11C, the waste maple syrup was demonstrated as a good alternative to cultivate micro-organisms, even if the production and productivity were slightly reduced compared to the control.

### Example 7: Use of Breweries' Waste as a Source of Nitrogen for the Production of Sophorolipids

Yeast extract are commercially marketed as powder, liquid or paste. They are generally derived from baker's or spent brewer's yeast (Saccharomyces cerevisiae in most cases) by yeast autolysis. With the aim of using alternative feedstocks, a alternative yeast extract sources were sought out. Several tests were conducted showing that spent brewer's yeast can be used as a suitable alternative source of nitrogen in the present fermentation process.

Spent brewer's yeast is available in large quantities and generally thrown away after several fermentation cycles by beer manufacturers. Although they represent a suitable feedstock meaning that they are relatively abundant and cheap, they are also known to contain undesirable components composed of hop resins and acids and beer solids from beer fermentation which prevent the yeast to properly grow. An optimized pre-treatment was therefore developed to get rid of the spent brewer's yeast bitterness and producing a yeast extract powder. Spent brewer's yeast were collected as a yeast paste and centrifuged (11,000 g-10 min-4° C.) to remove the beer liquor, resuspended in distilled water and adjusted to pH 9 with a NaOH solution for the debittering process. The solution is stirred at 4° C. for 30 minutes, centrifuged and washed several times with distilled water until the pH became neutral (usually three wash cycles until the pH went down to 7.7-7.5). The debittered yeast paste was then resuspended in 15% w/v distilled water and the pH was adjusted to 5.5 prior to autolysate the solution at 55° C. for 48 hours under constant shaking (150-180 RPM). After two days, the autolysis reaction was stopped by boiling the hydrolysate at 100° C. for 5 minutes. A final centrifugation step (11,000 g-10 min-4° C.) was performed to collect the supernatant containing all the water-soluble components including free amino acids, peptides, nucleotides, minerals, and vitamins (exact composition is unknown). Finally, the concentrate was spray dried and the resulting yeast extract powder was weighed out for its use as an alternative source of yeast extract. Flask assays were conducted in triplicate for two different source of brewer's yeast extract and compared to the commercially available yeast extract. These tests were realized using glucose (100 g/L) and waste fried cooking oil (10%) in addition to yeast extract 1 (YE 1=commercial yeast extract) and yeast extract 2 or 3 (brewer's yeast extract) at 5 g/L as feedstocks.

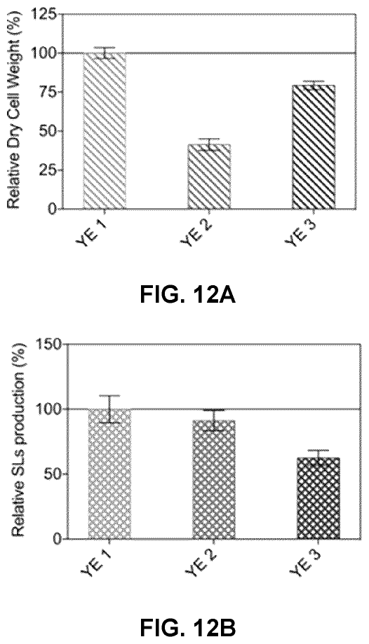

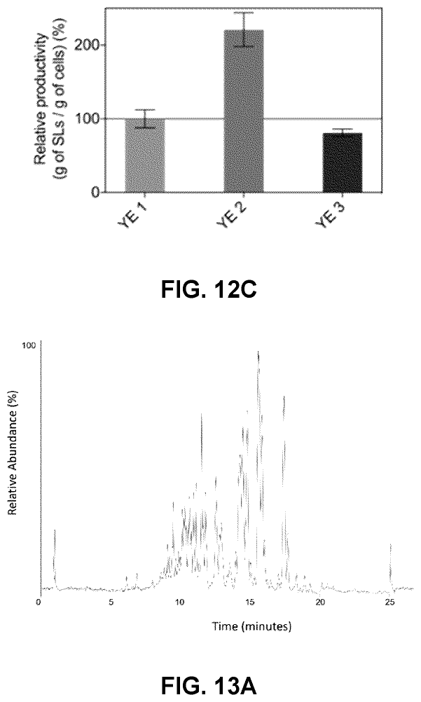

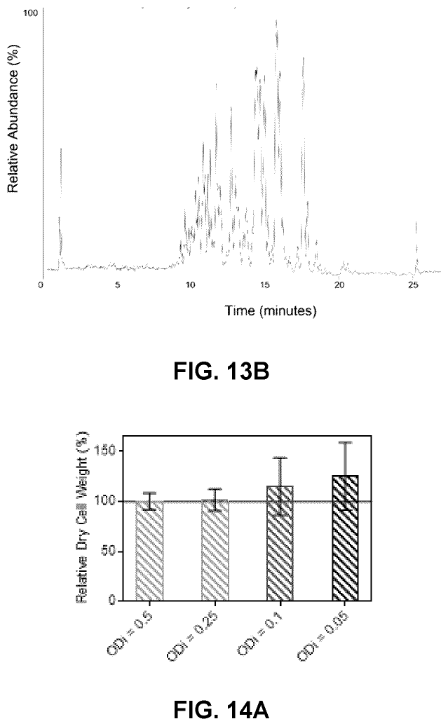

As shown in FIGS. 12A-12C, although the brewer's yeast reduce cell growth and resulting relative dry cell weight, the brewer's yeast maintained a similar sophorolipids production and therefore achieved an improved productivity. Moreover, the biosurfactant production profile during the production with the control commercial yeast extract (FIG. 13A) was found to be similar to the biosurfactant production profile of the tested waste yeast extract (FIG. 13B).

### Example 8: Impact of Inoculum Size on the Sophorolipid Production

The impact of the inoculum size on the growth and sophorolipids production was assessed. Several ODs were targeted for the beginning of the fermentation: 0.05, 0.1, 0.2 and 0.5. The growth was evaluated by dry cell weight and the sophorolipid production was measured by weighing the product after the extraction and evaporation processes. A 10% v/v fried cooking oil and pure glucose at 100 g/L as the carbon source were used for the fermentation.

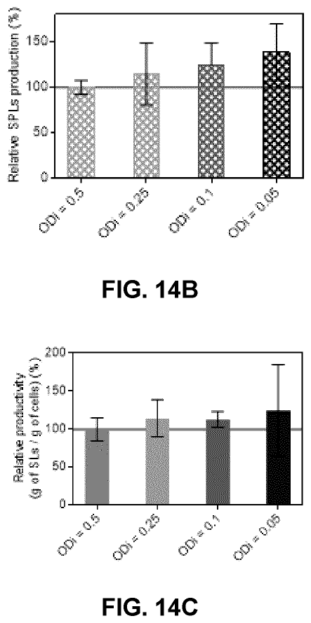

As can be seen in FIGS. 14A-14C, a lower OD, for example 0.05 provided more growth compared to 0.5 (FIG. 14A). The improved growth was accompanied by an increased production and a similar productivity (FIGS. 14B-14C).

### Example 9: Ethanol as the Extraction Solvent for Biosurfactants

Three fermentation flasks with about 23-24 mL volume of broth denoted as F1, F2 and F2 were obtained from a fermentation with

To these fermentation flasks, distilled water was added to bring the different media back to 25 mL (original starting volume) and the reactions were stopped by adding 3 vol/vol of ethanol to each flask (in this case, 75 mL of reagent grade ethanol was added to each flask).

Thereafter, broth and ethanol were vigorously mixed in a ratio of 1:3 vol/vol. The yeast was removed by centrifugation of the mixture at 4000 rpm for 10 min and the clear supernatant was analysed by high performance liquid chromatography (HPLC). Ethanol and water of the supernatant were removed by vacuum distillation (100 mbar pressure at 37° C.; it was observed that the vacuum required for the removal of ethanol was slightly lower than the vacuum required when ethyl acetate is used since the boiling point of ethanol is 58° C.).

The residue was suspended in two volumes of ethanol and filtered to remove polar components such as salt and sugar. The clear permeate was analysed by reversed phase-HPLC. The solvent was then removed by vacuum distillation and the residue weighed. Composition of this residue was determined using the relative peak areas of the corresponding chromatograms (Table 11).

CaCl₂) was used to aid the precipitation of oleic acid. CaCl₂) was added and incubated for three days at ambient conditions in the flasks. No precipitates were observed. The pH was lowered by acetic acid and in F3 some white precipitating pellets were observed.

TABLE 11
The three fermentation flasks (F1, F2 and F3).

| | F1 | | F2 | | F3 | |
|---|---|---|---|---|---|---|
| Deionized (DI) | 0.2 | mL | 0.7 | mL | 0.8 | mL |
| water added | | | | | | |
| Reagent ethanol | 75 | mL | 75 | mL | 75 | mL |

For all three flasks, centrifugation at 4000 rpm, 10 minutes (split
the 100 mL volume in 3 centrifuge tubes)
Cells settled at the bottom along with any unreacted oil (collect
the supernatant for vacuum evaporation) in all three flasks
The concentrate was filtered and analysed for salts and sugar for
all three flasks

| Lyophilized | 1.26 | | 1.16 | | 0.99 | |
|---|---|---|---|---|---|---|
| weight (g) | | | | | | |
| Water added | 10 | mL | 10 | mL | 10 | mL |
| Foaming | Foaming (fully | | Foaming (80% | | Foaming (80% | |
| observation | soluble) | | soluble) | | soluble) | |
| pH (native) | 2.93 | | 2.8 | | 2.92 | |
| pH adjusted | 11-12 | | 11-12 | | 11-12 | |
| (KOH solution) | | | | | | |

As can be seen therefore, the examples described above and illustrated are intended to be exemplary only. The scope is indicated by the appended claims.

### REFERENCES

Abdel-Mawgoud, A. M., Lépine, F., & Déziel, E. (2014). A stereospecific pathway diverts β-oxidation intermediates to the biosynthesis of rhamnolipid biosurfactants. Chemistry & biology, 21 (1), 156-164.

Alexis, J. (1995). Pratique industrielle de la méthode Taguchi: les plans d'expérience. Afnor.

Burch, A. Y., Shimada, B. K., Browne, P. J., & Lindow, S. E. (2010). Novel high-throughput detection method to assess bacterial surfactant production. Applied and environmental microbiology, 76 (16), 5363-5372.

Funston, S. J., Tsaousi, K., Rudden, M., Smyth, T. J., Stevenson, P. S., Marchant, R., & Banat, I. M. (2016). Characterising rhamnolipid production in Burkholderia thailandensis E264, a non-pathogenic producer. Applied Microbiology and Biotechnology, 100 (18), 7945-7956.

Gibson, D. G., Young, L., Chuang, R. Y., Venter, J. C., Hutchison III, C. A., & Smith, H. O. (2009). Enzymatic assembly of DNA molecules up to several hundred kilobases. Nature methods, 6 (5), 343-345.

Martinez, S., Humery, A., Groleau, M. C., & Deziel, E. (2020). Quorum sensing controls both rhamnolipid and polyhydroxyalkanoate production in Burkholderia thailandensis Through scmr regulation. Frontiers in bioengineering and biotechnology, 1033.

Morsi, H., Yong, K. L., & Jewell, A. P. (2004 December). Evaluation of the Taguchi methods for the simultaneous assessment of the effects of multiple variables in the tumour microenvironment. In International Seminars in Surgical Oncology (Vol. 1, No. 1, pp. 1-9). BioMed Central.

Nickzad, A., Guertin, C., & Déziel, E. (2018). Culture medium optimization for production of rhamnolipids by Burkholderia glumae. Colloids and Interfaces, 2 (4), 49.

Smith, D. D., Nickzad, A., Déziel, E., & Stavrinides, J. (2016). A novel glycolipid biosurfactant confers grazing resistance upon Pantoea ananatis BRT175 against the social amoeba Dictyostelium discoideum. Msphere, 1 (1), e00075-15.

1. A Burkholderia thailandensis DIS2 bacteria as deposited at IDAC under Accession No. 240222-01, a mutant or variant thereof.

2. A bacteria having all the identifying characteristics of Burkholderia thailandensis DIS2, deposited at IDAC under Accession No. 240222-01, a mutant or a variant thereof.

3. A Burkholderia thailandensis DIS2.1 bacteria as deposited at IDAC under Accession No. 070323-01, a mutant or variant thereof.

4. A bacteria having all the identifying characteristics of Burkholderia thailandensis DIS2.1, deposited at IDAC under Accession No. 070323-01, a mutant or a variant thereof.

5. A Starmerella bombicola DIS4 yeast as deposited at IDAC under Accession No. 210223-01, a mutant or variant thereof.

6. A yeast having all the identifying characteristic of Starmerella bombicola DIS4 deposited at IDAC under Accession No. 210223-01, a mutant or variant thereof.

7. A method of producing a rhamnolipid, the method comprising: performing a fermentation with Burkholderia thailandensis, a mutant or variant thereof, wherein the fermentation is performed with a feedstock comprising a waste carbon source, and fried cooking oil.

8. The method of claim 7, wherein the waste carbon source is enzymatically treated, physically treated, heat treated or untreated.

9. The method of claim 7, wherein the waste carbon source is selected from the group consisting of whey, glycerol, maple syrup waste and starch waste.

10. The method of claim 7, wherein the feedstock further comprises ethanol.

11. The method of claim 7, wherein the Burkholderia thailandensis is Burkholderia thailandensis DIS2 and/or Burkholderia thailandensis DIS2.1.

12. The method of claim 7, wherein the fried cooking oil is selected from the group consisting of fried corn oil, fried canola oil, fried peanut oil, fried coconut oil, fried grapeseed oil, fried soybean oil, fried olive oil and fried cottonseed oil.

13. The method of claim 7, further comprising extracting the rhamnolipid from a culture media of the fermentation by a solvent extraction with an extraction solvent.

14. The method of claim 13, wherein the extraction solvent is ethyl acetate or ethanol.

15. A method of producing a sophorolipid, the method comprising: performing a fermentation with Starmerella bombicola DIS4 yeast as deposited at IDAC under Accession No. 210223-01, a mutant or variant thereof, wherein the fermentation is performed with a feedstock comprising a waste carbon source, and fried cooking oil.

16. The method of claim 15, wherein the waste carbon source is enzymatically treated, physically treated or heat treated or untreated.

17. The method of claim 15, wherein the waste carbon source is selected from the group consisting of whey, glycerol, maple syrup waste and starch waste.

18. (canceled)

19. The method of claim 15, wherein the fried cooking oil is selected from the group consisting of fried corn oil, fried canola oil, fried peanut oil, fried coconut oil, fried grapeseed oil, fried soybean oil, fried olive oil and fried cottonseed oil.

20. The method of claim 15, further comprising extracting the sophorolipid from a culture media of the fermentation by a solvent extraction with an extraction solvent.

21. The method of claim 20, wherein the extraction solvent is ethyl acetate or ethanol.


SEQUENCE LISTING (12 sequences)

SEQ ID NO: 1 - DNA, 30 bp, synthetic construct
  source 1..30: mol_type = other DNA
tttccaacccgttggatccccgggtaccga

SEQ ID NO: 2 - DNA, 24 bp, synthetic construct
  source 1..24: mol_type = other DNA
agttttgtgcaataccaaccgacg

SEQ ID NO: 3 - DNA, 24 bp, synthetic construct
  source 1..24: mol_type = other DNA
cgtcggttggtattgcacaaaact

SEQ ID NO: 4 - DNA, 32 bp, synthetic construct
  source 1..32: mol_type = other DNA
cgaggaccattccgaaagcttggcactggccg

SEQ ID NO: 5 - DNA, 37 bp, synthetic construct
  source 1..37: mol_type = other DNA
ccagtgccaagctttcggaatggtcctcgaagaagag

SEQ ID NO: 6 - DNA, 46 bp, synthetic construct
  source 1..46: mol_type = other DNA
tctagagtcgacctgcaggcatgcaagtaaactgacgcgcaccttt

SEQ ID NO: 7 - DNA, 30 bp, synthetic construct; identical to SEQ ID NO: 1
  source 1..30: mol_type = other DNA

SEQ ID NO: 8 - DNA, 24 bp, synthetic construct
  source 1..24: mol_type = other DNA
agttttgtgcaataccaaccgacg

SEQ ID NO: 9 - DNA, 1276 bp, Starmerella bombicola
  source 1..1276: mol_type = genomic DNA
agatgggaaggctggaaatgcagccggtagccgaacgactggttggcggcatcgaacgaatcgtggtcgtactgccggctgctgaaaagaatcacgcgcacgcttggcctccggtaaaggtgcgcgtcagtttacttcagccgggcgtcactggagcccggcgacgacgtccgggcgcttcgccgtgccggcttcgcgcatgaagtagtggccgttcgtctcggtcttctgcccgccgaggtggtacagcgcgtgctcgacgaagctgcgcgtgcgcgccggcaggaactggcggttcggatagacgatggacaactgcgtgtcgggatcgtcgatccggtaatcgccgagaatccgaacgagctcgccgctttcgagcgcatcggcgatgtagctttccggcagcacggagatgccggtgccggcgagcgtcgccgcgcgcaccatcgacgcgctgttgaccgtatagaccggctgcagcgacacgacgtgcgtgaagtggtccggatcgacgaagcgccaggtcgatgcatgctggccggccggcagcgcgatgcacgcgtggcggatgagatcgtcggggcggtgcggcgcgccgtgccgctcgatgtacgagggcgccgcgcagagcgcgagcgtgttcggcatcagcggatgaccgatgagcgccgggttgccgtcgaggcggccgcccgtgacgaggcccgcgtcgtagccggcttcgatcacgtcgagctgcccctcggtcagcgtcaactgcacgcgcaccttcgggtactggcgccggtagctgtcgacgagcggggtgatccggccgggcgacagcagcccggacacggcgacgcgcaacgtgccgacgggctcgtgcaccgctcgctcgacggacgcctcgagctgatcgaattcctcgaggagcgcccggcagccgtcgaggtagcggatgcccgcttccgtcagcgacagattgcgtgtcgtgcgatggatgagacgcgtgttcagatgtgtttcgagcatcgcgatcgaacgcgtgacgagcgcattcgatacgccgagtttccgcgccgcccggcggaagctctgcagttcggcaacacatacgaagacacgcatggtctggatttggttcatagctttcgttggcccattaaatgattatgttgtgtttttcgccttcgatcatggcgagccgcggctgtcggatcagcacagggcttgtcttaaataacgaatcatcgaaggctgggcaaattgttcaataagctgacagcatcagtca

SEQ ID NO: 10 - DNA, 279 bp, synthetic construct
  source 1..279: mol_type = other DNA
nnnggggcgcagaggttggggctcgacgatcgtggtcgtactgccggctgctgaaagaatcacgcgcacgcttggcctccggtaaaggtgcgcgtcagtttacttgcatgcctgcaggtcgactctagaagctttcgttggcccattaaatgattatgttgtgtttttcgccttcgatcatggcgagccgcggctgtcggatcagcacagggcttgtcttaaataacgaatcatcgaaggctgggcaaattgttcaataagctgacagcatcagtcaaa

SEQ ID NO: 11 - DNA, 24 bp, synthetic construct
  source 1..24: mol_type = other DNA
gcatatcaataagcggaggaaaag

SEQ ID NO: 12 - DNA, 19 bp, synthetic construct
  source 1..19: mol_type = other DNA
ggtccgtgtttcaagacgg